(19) United States
(12) Patent Application Publication
(10) Pub. No.: US 2006/0015142 A1
(43) Pub. Date: Jan. 19, 2006

(54) Plug made of mesh material for closing large trocar wounds

(76) Inventor: Zafer Malazgirt, Samsun (TR)

Correspondence Address: ELLIS & VENABLE, PC, 101 NORTH FIRST AVE., SUITE 1875, PHOENIX, AZ, 85003, US

(21) Appl. No.: 10/527,365

(22) PCT Filed: Feb. 27, 2003
(86) PCT No.: PCT/TR03/00014

§ 371 Date: Mar. 9, 2005

(30) Foreign Application Priority Data
Sep. 13, 2002 (TR) 2002/02198

(51) Int. Cl. A61B 17/08

(52) U.S. Cl. 606/213

(57) ABSTRACT

The invention is about a material and its relevant technique that is used in the repair of large trocar wounds. The method as a whole is simple to use and minimizes tissue trauma. With this invention, the rest of the suturing techniques are set aside, and by the use of a specially designed plug, the trocar wound repairing process is reduced to a simple and quick procedure. The material is basically made of polypropylene, of which a specific layer covers the intraabdominal face. The material functionally consists of three pieces, and can be called as plug and mesh. The plug and mesh can be used in the repair of almost all large trocar fascial defects that occur at laparoscopic surgery. Due to its unique design, the plug and mesh can fix most of these large trocar wounds safely and securely.

### TECHNICAL AREA

The invention is about a plug and mesh material that is to be used in the closure of large trocar wounds without any need for suturing after laparoscopic operations.

### BACKGROUND OF THE INVENTION

Trocar site herniation is a recognized complication of laparoscopic surgery. Omental and-sometimes-intestinal herniation with incarceration and obstruction has been documented in recent surgical literature, occurring particularly at 10 mm or larger trocar sites that were not sutured at operation. The necessity to perform fascial closure of any trocar insertion site greater than 5 mm has now been established and is routinely practiced worldwide.

However, the conventional closure of such a trocar site fascial defect is often technically difficult, frustrating, indefinitely successful, and even sometimes dangerous due to the limited size of skin incision, the depth of the subcutaneous fatty layer, and necessity of blind manipulation. Moreover, the suturing that involves placement of deep blind sutures after the abdomen has been decompressed is a dangerous manipulation that surgeons tend to avoid.

A number of techniques and instruments have been suggested in the recent 8 to 10 years to facilitate a safe and secure closure of the fascial defect through the tiny skin opening. All of these repairs include passing in any way a suture from one side of the trocar wound to the other, and its ligation. For this purpose either a heavy needle or a variety of straight needles through which sutures are passed have been used.

Stringer (1995) utilized a Grice needle while Garzotto et al (1995) used a spring-loaded needle. Weiland et al (1995) proposed the use of a Veress needle to pass the sutures through. Robertson (1996) reported a similar method. However, in his method he used two different sutures, of which the ends were taken out through the trocar, and were tied outside, and then pulled outside by pulling one of the sutures. Hellinger et al (1996) have recommended a self-contained disposable fascial closure device. Schwegler et al (1996) described a hook-needle, Thevissen and Pier (1996) reported trocar site closure utilizing the Carter-Thomason technique, and Ammori (1996) recommended the use of a 110-mm heavy spatulated suture needle. In Ammori's method, the ends of a suture, which penetrated the abdominal wall from skin to skin were brought out of the subcutaneous fat tissue by the help of a nerve hook. Petrakis (1999) recommended a meticulous purse string technique for the closure of trocar defects. Chapman (1999) described the Gore Suture Passer, which are both a needle and a suture grabber.

The basis of the above-mentioned reports can be summarized as that the trocar wound is somehow repaired by any of the suture techniques. Although described in most of the reports as easy and quick methods, these suturing techniques require positioning of the camera and graspers, visualization of the needles during their entrance into the peritoneal cavity, feeding of the graspers or suture passers with the suture loop, all of which are repeated once to thrice for every trocar defect. Any of these suturing techniques are not only time and effort consuming, but also require sophisticated laparoscopic talent and coordination. As more defects at various sites in the abdominal wall are to be closed after advanced laparoscopic operations, the laparoscopic procedures that support the suturing techniques become more complicated and complex. The above-mentioned suturing techniques would therefore be not that easy and quick.

Moreover, a series of manipulation is needed to complete a single suturing. The conventional suturing technique involves much traumatic manipulation including pushing, pulling and retraction of the wound, and insertion and extraction of needles. Most of the time the needle is passed twice, and sometimes more (as depicted in Petrakis' technique). As manipulation in the wound increases, the inflammation and risk of ensuing infection rise considerably. The edema and the collection of seroma and hematoma at the wound further cause dehiscence and hernia formation on a long-term basis.

Excessive traumatic manipulation and suturing with heavy sutures oppose the “minimal damage” basis of laparoscopic surgery. The patients are subject to pain and complications at their trocar sites in the postoperative period. The problems associated with the repair of trocar wound would be annoying to the patient as he (or she) is discharged on the first or second postoperative day. The problems of the wound would cause the patient to refer back to the institution.

Any of these suturing techniques are to be done under direct vision. It is however impossible to repair the last trocar wound under direct vision. Unless a 0.5 cm scope is used, the last large trocar site can only be closed with conventional blind sutures. At a regular laparoscopic cholecystectomy, the surgeon can only repair the first of two large trocar defects under direct vision. He must close the last one blindly.

No matter which suturing technique or needle is used, it is not possible to eliminate the trocar site hernias completely. The current incidence is reportedly around 0.77-3%. As complex-laparoscopic surgery becomes more common, the incidence of this complication increases. The reported rates of hernia show that there is not yet any superior method in the safe closure of the trocar fascial defect.

In 2002 Rosin et al advocated closure of the trocar wound by fibrin glue. Their method differs from the previously reported techniques in the following respects. It consists of no sutures, and that it coincides with the “minimally” invasive nature of laparoscopy. The glue can obliterate the subcutaneous tissue and close the skin. However, it seems unrealistic to think that fibrin glue can securely fix the fascial opening.

### DETAILED EXPLANATION OF THE INVENTION

The invention is about a material and its relevant technique that is used in the repair of large trocar wounds. The method as a whole is simple to use and minimizes tissue trauma. With this invention, the rest of the suturing techniques are set aside, and by the use of a specially designed plug, the trocar wound repair is reduced to a simple and quick procedure.

The material is made of polypropylene, of which a specific layer covers the intraabdominal face. The material functionally consists of three pieces, and can be called as plug and mesh. This 3-D plug and mesh material agrees well with the rules of hernia surgery, is appropriate to the “minimally invasive surgery” basis of laparoscopy, and is friendly with the physics laws effective on the anterior abdominal wall.

The plug and mesh can be used for the repair of almost all large trocar fascial defects that occur during laparoscopic surgery. Due to its unique design, the plug and mesh can fix safely and strongly into most of the large trocar wounds.

Since its use does not necessitate “direct vision”, it does not differ whether it is to be used at the first or last trocar wound. Direct vision from an intraabdominal aspect by a laparoscope during the insertion and positioning of the plug and mesh is not necessary. The exploration of the wound from outside before or during its insertion is not required, as well. At the beginning of the operation every trocar site is examined by the laparoscope in respect with the availability of positioning of a plug and mesh. Except the first, the trocar sites in a laparoscopic operation are spontaneously visualized anyway as the insertion of the trocars into the peritoneal cavity are routinely followed by the laparoscope. Most of the trocar sites are flat, smooth and clean that only a few necessitate minor and very brief preparation. Therefore there is no need for interior or exterior direct viewing during the insertion of the plug and mesh. To summarize, there is no need to switch to a blind suturing technique during the repair of the last trocar wound. The plug and mesh is inserted into place by a simple maneuver. Its proper positioning in the place and the fixation of the ears to the outer fascia are easily accomplished by the guidance of a clamp and fingertip.

It is easy to use, and its insertion and positioning is very simple and quick. It does not necessitate exhaustive retraction and manipulation. The insertion and placement of the plug in the wound approximately takes less than a minute. If repeated twice in a standard laparoscopic cholecystectomy or more in an advanced procedure, it lowers the operating time by minutes. The fact that the surgeon repairs the defects easily, quickly and safely contributes to a pain-free healing of the wound and causes fewer complications.

The plug itself and its insertion and positioning in the wound are natural safety measures against the so-called “chimney effect”. The chimney effect is the unwanted escape of the bowel or omentum through the trocar wound during or after the deflation of the pneumoperitoneum. During blind suturing, this effect may cause dangerous complications. The insertion of plug and mesh sweeps away and draws back any herniated intraabdominal structure, and prevents any further herniation thereafter.

It conceals the intraperitoneal orifice (inner ring) and fits perfectly into the trocar's tunnel in the abdominal wall. The specially designed neck of the plug and mesh initially gives a better hold in the wound. The special shaggy design activates in-growth of the granulation tissue, which further stabilizes the plug in place, and facilitates its better and faster incorporation in the trocar defect.

It is a pressure-friendly material. It functions in company with the physics laws and intraabdominal pressure. In suturing techniques, there is a force-resistance counter-relation between the suture material and tissue tension and pressure. There is no such problem with plug and mesh. The intraabdominal pressure holds the mesh in place, and secures the integrity of the repair. Tissue tension has no detrimental effect on a plug and mesh. Because of Pascal Law, the appropriate surface area of the mesh prevents its prolapse into the defect. Contrary to suturing, the plug and mesh has no physical effects like squeezing, pulling or pushing. This feature contributes to earlier return of the patient to his (or her) normal daily activity, and enables him-to resume sporting activities before long. This is a fact that conforms well to the basic initiative of the no-tension hernia repair techniques. I specifically recommend its use in patients with increased intraabdominal pressure. Its harmony with Pascal Law makes it a superior choice in patients with debilitating diseases. Its use is especially indicated after laparoscopic surgery in those patients with high intraabdominal pressure (due to ascites, CAPD application in chronic renal failure, and obesity).

The plug and mesh causes less wound problems in the early postoperative period. Less tissue trauma decreases the incidence of seroma, hematoma, inflammation, infection and dehiscence, per se. We believe that the figures of hernia incidence would decline by the use of this material, as well.

The standard skin stapler or a specially designed stapler can easily fit into the wound through the skin opening. Guided by the fingertip, the ears of the plug and mesh are stapled onto the outer fascial layer one at a time. One staple to each of the two ears of a plug fixates the material in place, and prevents its slippage into the abdominal cavity in any circumstance. A clip applier may well be used for this purpose, as well. The clip applier with a partially projected clip at the end is introduced into the place with an almost horizontal angle. It is fired while one of the prongs of the clip is trusted through the mesh and under the fascial layer. The closed clip holds a few strands of the mesh and the fascia together. Two clips are to be put in place if this method is used. Gluing of the ears to the fascia and no fixation at all are other alternatives.

The plug and mesh is made of polypropylene material, a material that causes intraabdominal adhesions if laid in close proximity to intestines or other intraperitoneal structures. An antiadhesive layer should therefore precover the intraabdominal face of the mesh plate. Today there are three composite mesh materials produced by three different companies who have this antiadhesive layer on one face of the material. Namely, these are Parietex Composite (Sofradim International), Sepramesh (Genzyme) and Composix (Bard). All three materials are made of polypropylene. However, their antiadhesive layers differ. The antiadhesive layer in the first two is absorbable collagen barrier, and ePTFE in the latter. New composite mesh products are expected soon.

The intraabdominal mesh plate must always lie flat and fully open. It must not be wrinkled or folded, up on itself, and it must never come in contact with the intestine in a perpendicular fashion. Such a contact may cause intestinal fistula, and lower its impact on hernia prevention. The special design of the plug and mesh, namely the legs holding on the mesh plate at, its periphery, enables the edges to be pulled towards the abdominal wall, and prevents the edges from coming in contact with the intestine.

Creation of a socket is to be formed preferably right after the insertion of the trocar if a smooth and clean intraperitoneal surface surrounding the inner trocar hole is not present. At a standard laparoscopic cholecystectomy, preparation of such a socket may be necessary at the upper median trocar site, and can easily be formed in seconds after insertion of the median epigastric trocar. For this purpose a short longitudinal incision along the root of the falciform ligament and minimal dissection around is made. The pneumoperitoneum further blows it open during the procedure, and the socket is ideally formed as the time for insertion of a plug and mesh comes.

The material, as a whole, looks like a reverse T. It can be best described as a “plug and mesh”, and is comprised of a horizontal plate and a vertical plug (FIG. 1). However, it is a union of three functionally distinctive pieces (FIG. 2).
1. The intraabdominal mesh plate
2. The shaggy neck piece.
3. The double-ear piece.
   Intraabdominal Mesh Plate:

It is made of polypropylene, and either a hydrophilic absorbable collagen barrier antiadhesive or a layer of ePTFE covers its obverse face. It is round in shape. If pulled gently, it resists to be herniated into the trocar wound, and will have a convex shape as seen from an intraabdominal laparoscope. If not pulled anymore, it flattens itself from a more convex to a less convex position. These two characteristics of the mesh plate are obtained either by a double layer of polypropylene sheet or by a thick (1 mm) single layer polypropylene sheet.

It has a diameter of 4.5 cm. At calculations we assumed that the inner trocar hole to be 1.0 cm² in size. This size will prevent its prolapse into the trocar wound even if the pressure at the trocar site increases by 20 times while it is normal at other points on the mesh. The intraabdominal mesh plate is inserted into the abdominal cavity through the trocar channel, and is laid flat open between the abdominal wall and the intestines centering the inner trocar hole.

Pascal Law says that if we apply a pressure to some part of the surface of a confined fluid by means of a piston, then this pressure will be transmitted without change to all parts of the fluid. Due to the biological properties and consistency of the organs, we can assume that the intraabdominal medium behaves more or less similar to liquids. Increased intraabdominal pressure will be distributed almost unchanged to all points on the abdominal wall.

According to the Pascal taw, the pressures at two pistons are equal. However, the force is directly correlated with the surface area of the piston. Normally, the pressure on the inner trocar hole is similar with the pressure at different points on the mesh, a fact that prevents the herniation of the mesh into the trocar channel.

Let us consider now an odd condition that the pressure on the inner trocar hole is increased to a peak of 20 times that of other points on the mesh. In order to prevent prolapsing of the mesh into the trocar tunnel, the total force on the mesh must be higher than or equal to the force on the inner trocar hole.

| | |
|---|---|
| ΔP = ΔF/ΔA | ΔP: pressure at a certain point |
| ΔPₜₕ = Δₚₛ | ΔF: force at a certain point |
| ΔFₜₕ/ΔAₜₕ = ΔFₚₛ/ΔAₚₛ | ΔA: area influenced by the force |
| ΔFₚₛ = 20. ΔFₜₕ | th: trocar hole |
| ΔFₜₕ/ΔAₜₕ = 20ΔFₜₕ/ΔAₚₛ | ps: plate surface |
| ΔAₚₛ = 20. ΔAₜₕ | |

Since the mesh is small, in size, πr² will, be used in the calculation.
πrₚₛ²=20.πrₜₕ²
rₚₛ²=20.rₜₕ² (rₜₕ=0.5 cm)
rₚₛ²=20×0.25 cm²
rₚₛ²=5.0 cm²
rₚₛ=2.24 cm≅2.25 cm
Rₚₛ=4.5 cm.

This final figure shows what diameter the plate should be. For bigger trocar holes, the size of the plate must be recalculated accordingly.

The Shaggy Neck Piece:

This piece, which is made of polypropylene, connects the plate to the double-ear piece. Its specially designed shaggy structure helps in taking a good hold of the trocar tunnel.

It is a cylinder with a diameter and height of 1 cm. The shaggy appearance comes from the projecting loops of polypropylene very similar to those of a heavy towel. The loops are 2 mm in length. Thus the diameter of the neck reaches to 14 mm in total. Towards bottom the neck cylinder opens up to form four legs, all of which unite with the plate's periphery on the reverse side. The shaggy neckpiece continues with the two ears above.

The bottom of the cylinder extends to form four legs at an angle of 100°. Every foot is 1.3 cm in length and 0.6 cm in width. All the feet merge with the plate at its periphery with 0.3 cm segments at the tip (FIG. 3). When the ears are pulled upwards too much or too strongly, the feet pull up the plate from its periphery. Thus the circumference of the plate are collected upwards, inwards and away from the intestine giving a convex shape to the abdominal plate. If these feet are not existing, and the neck pulls the plate directly from its center, then the plate will be pulled upwards while bending the ends down towards the intestine. This causes perpendicular contact of the edges with the intestine. This is the most unwanted position of the mesh.

Above, the shaggy neck cylinder loses some of its circumference symmetrically, and continues upwards to form two reciprocal ears. The circumference of the neck cylinder is 3.14 cm. The radix of each ear is 1.0 cm in width. There are two 0.7 cm bare neck edges between the ears. The shaggy neck piece is made of polypropylene, and its heavy textured style or its shagginess is obtained either one of two methods. First, a heavy texturing style is adopted at this segment, or second, a heavy-textured rectangular segment measuring 3.15 cm by 1.1 cm (the overlapping margins are excluded) is wrapped around the neck piece, and fixated accordingly.

Double-Ear Piece:

Although the ears are mentioned as functionally different pieces, they are, in fact, materially undivided from the shaggy neck piece. The ears extend upwards uninterruptedly from the neck cylinder edge, stand up parallel, and face to each other. Each ear is 1.0 cm in width and 3.0 cm in length from the edge of the neck cylinder. It reaches to a width of 1.4 cm at the tip. At its radix its shape is concave, but becomes flat superiorly. This concavity gives the material an extra holding strength in the trocar tunnel. If the distal half to one-third of an ear is bent outwards by the help of fingers, the tip holds its bent position, a fact that is derived both from the physical property of the material and the concavity at the radix. Since the length of the trocar tunnel from the peritoneum to the outer surface of the fascia varies moderately among patients and trocar sites, the ears contribute to the adjustment of the plug piece in every trocar tunnel.

How is the Material (Invention) Used?

At the end of any laparoscopic operation, the surgeon decides on which trocar wounds are to be repaired by the plug and mesh. The surgical nurse should prepare the equivalent number of plug and mesh material and the “multipurpose stapler” (if available) at her table.

Using the laparoscope, the surgeon rechecks the inner hole whether it has a clean flat area around with a radius of 2.5 cm. Any hole, around which a sufficient clean flat area is impossible to develop, must be excluded from the plug and mesh repair.

It is easier to place a plug and mesh material in the presence of pneumoperitoneum, however this is not essential. It can be used safely after complete deflation, as well. If the surgeon wants to keep the pneumoperitoneum before and after insertion of the first plug, one of his (or her) assistants must employ a finger (FIGS. 4b, 4c). If not done so, some gas will eventually escape through the mesh.

Insertion of a Plug and Mesh does not require any form of direct vision. However, inner vision of the insertion and placement of the mesh plate can be visualized by the laparoscope if it is in the abdomen and not used for any other purpose. In the first few cases, the surgeon may not exactly know with how much force he should pull the ears up. It is helpful to the surgeon in this initial phase for developing a sensation of an ideal pullback tension. After the first few cases, insertion and positioning of the material can easily be accomplished blindly. The last trocar wound to be repaired must preferably be the umbilical one.

The subcutaneous tissue above the outer fascial layer adjacent to the trocar tunnel is slightly dissected by a middle-sized clamp to form two small gaps (FIGS. 5a, 5b). During this step the size of the gaps must be memorized for the next step, at which point the surgeon must decide whether he should trim the ears. The direction of the gaps must be parallel to the fascial tension lines. In the anterior abdominal wall, the gaps are placed horizontally.

A heavy curved clamp holds the plug and mesh in such a position that the ears are on top of each other (FIG. 6a). The tip of the clamp is exactly at the neck-ear junction. The nurse soaks the material into saline solution. As the assistant pulls away his finger, the surgeon inserts the clamped material into the wound, and gently pushes it with a steady jerk until the bulk gets into the abdominal cavity (FIG. 6b). The resistance suddenly disappears as its entire length enters into the abdominal cavity. Pushing it further to various directions or to-and-fro movements in the abdomen is not recommended. At this point the surgeon pulls back the clamp until he sees in the screen or feels at his hand that the mesh plate is touching the parietal peritoneum slightly. After checking that the tips of the ears are out of the fascial layer, the clamp is taken out. The ears are brought all the way out of the skin (FIG. 8a).

A clamp checks the gaps that were prepared beforehand. The ears are trimmed accordingly. The trimmed tip of an ear is held by a middle-sized clamp, and pushed inside and to the gap settling onlay on the fascia (FIG. 8b). The surgeon's index fingertip helps accomplishing this step very easily. The same is repeated for every trocar site.

If a “multi-purpose” stapler is available, it is inserted into the gap, and is shot to put down a titanium clip over the mesh to the fascia. Ideally, the clip should be put at outer one-third segment of each ear.

As the ear is put into its gap, the fingertip checks its proper position. With the finger in position, the stapler may be pushed in and fired. If both cannot fit into the hole, then stapler is pushed while the fingertip is pulled back. The stapler is fired halfway to let the clip appear at the nozzle. The stapler then tackles the mesh, and both are stretched laterally, and the stapler is fully fired. The same is repeated on the other side.

### SHORT DESCRIPTIONS OF THE FIGURES

FIG. 1: The perspective appearance of the material

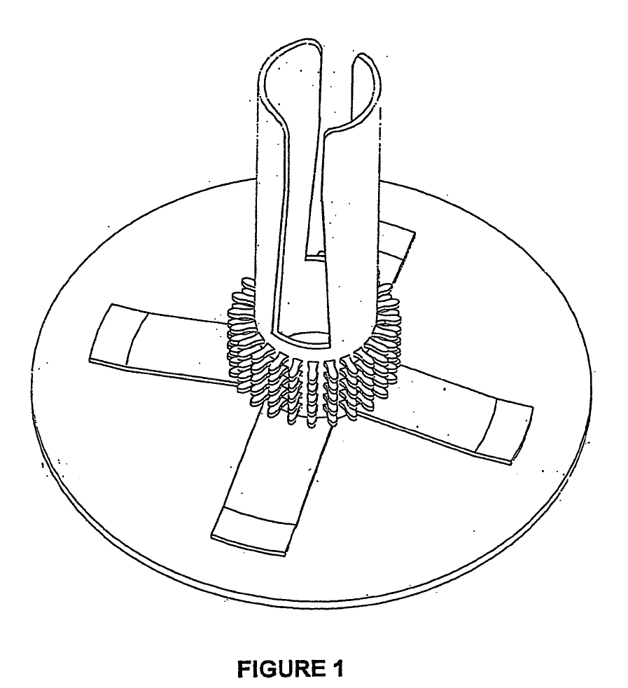

FIG. 2a: Side view of the plug and mesh from one angle.

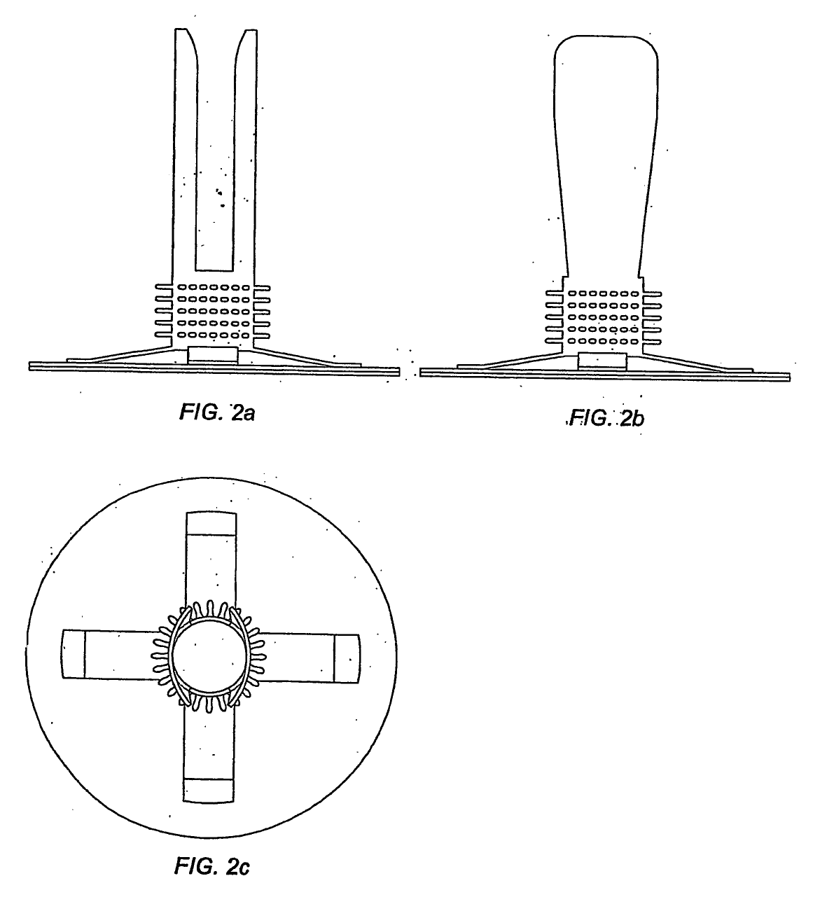

FIG. 2b: Side view of the plug and mesh from another angle.

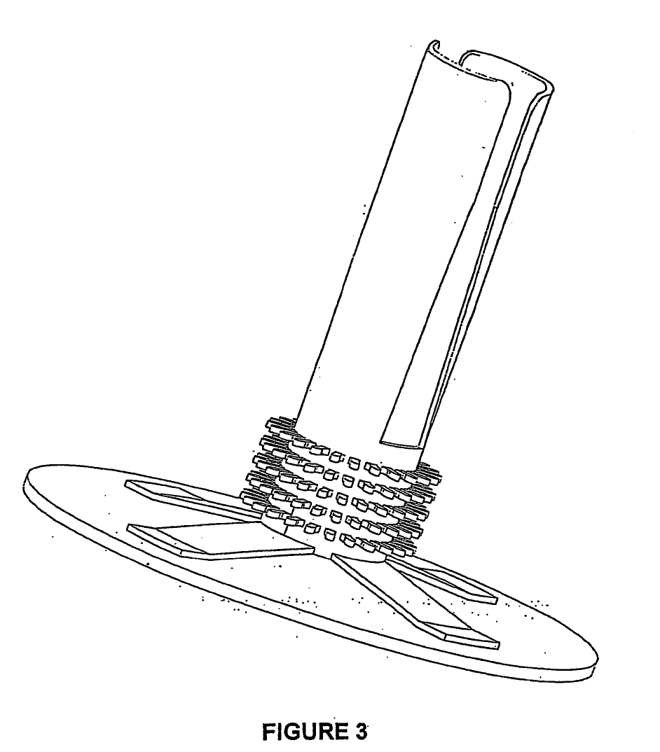

FIG. 2c: View of the plug and mesh from above.

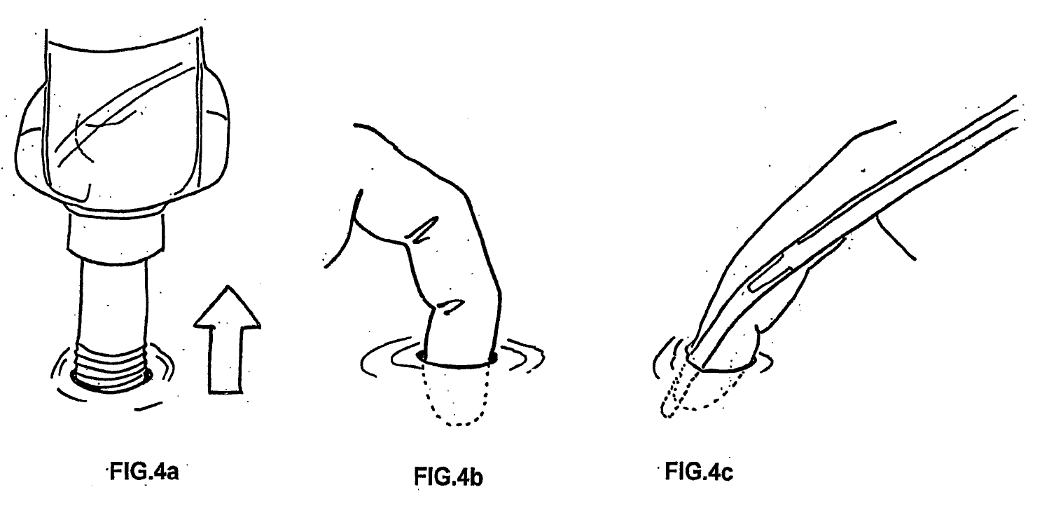

FIG. 3: The perspective appearance of the material depicting the stretching of the legs and their union with the periphery of the mesh plate.

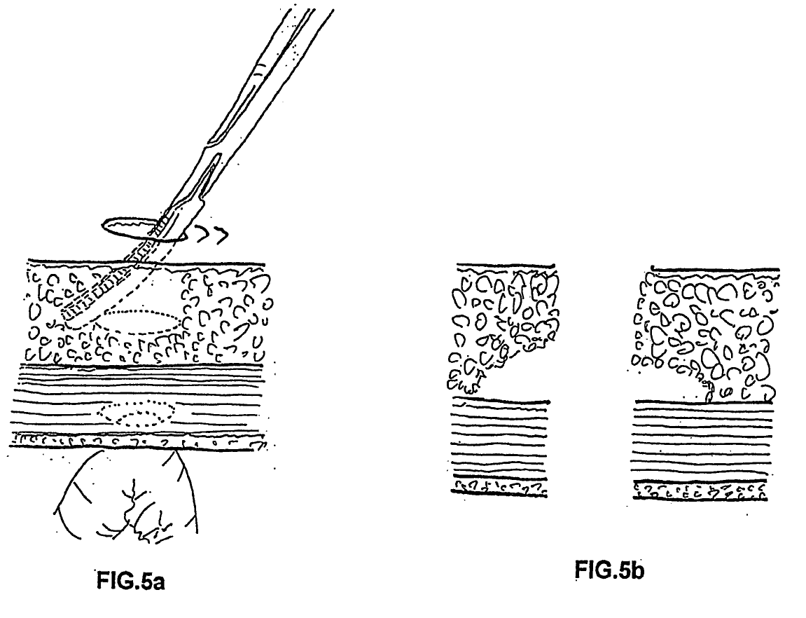

FIG. 4a, 4b, 4c: Extraction of the trocar at the end of the primary procedure, insertion of the finger into the wound to halt escape of the gas, and exploration of the trocar tunnel by a heavy clamp.

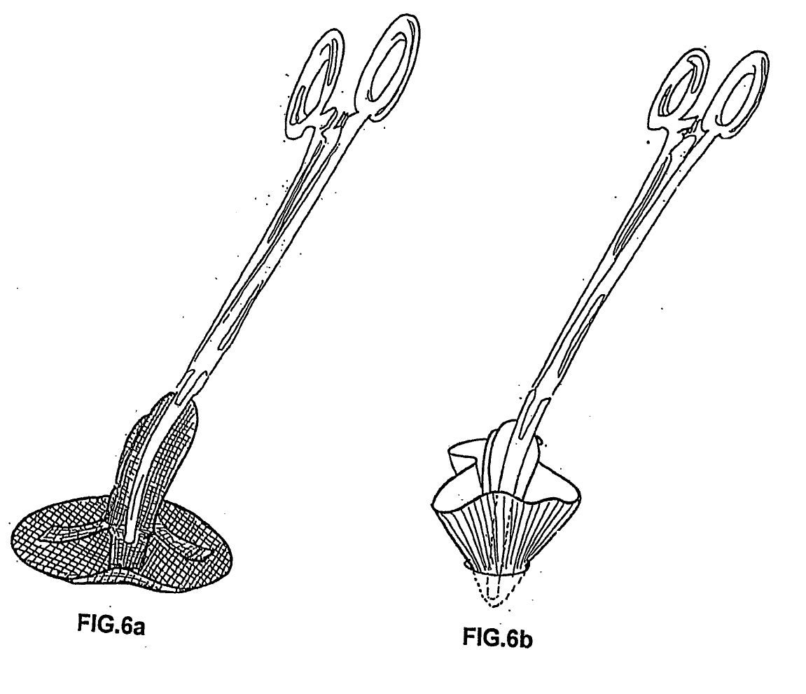

FIG. 5a, 5b: The making of a gap in the subcutaneous tissue, in which thereafter the double ear piece (3) will be settled in.

FIG. 6a, 6b: The insertion of the mesh and plug in the trocar hole. Note that the clamp holds the material at its double ear segment (3).

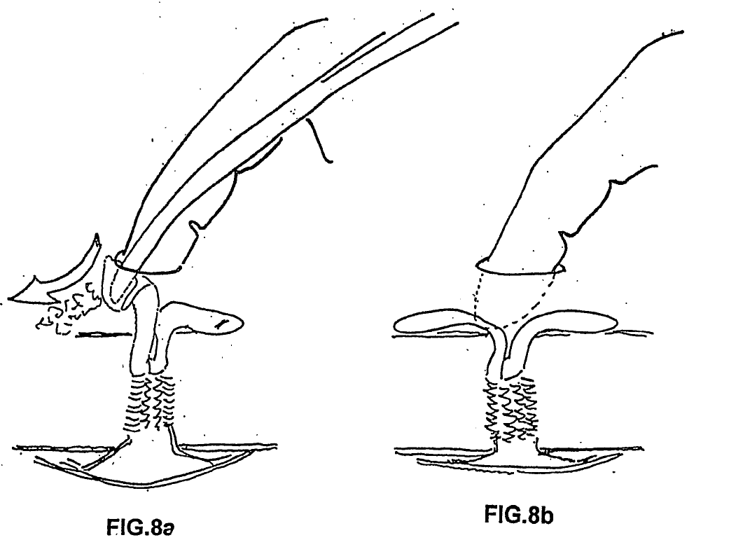

FIG. 7a, 7b: The positioning of the mesh plate (1) over the inner trocar hole by pulling the double ear piece (3) upwards.

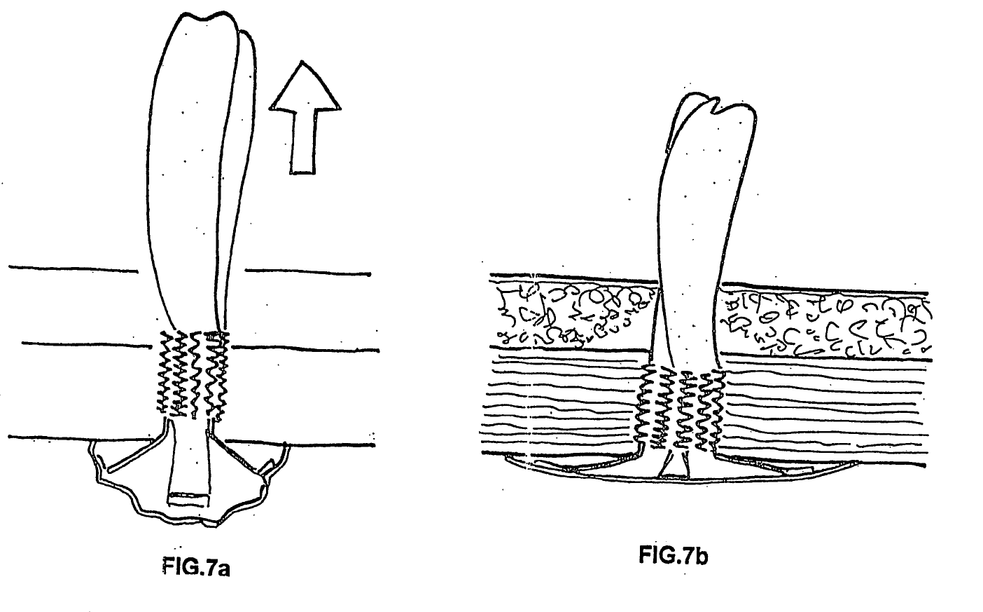

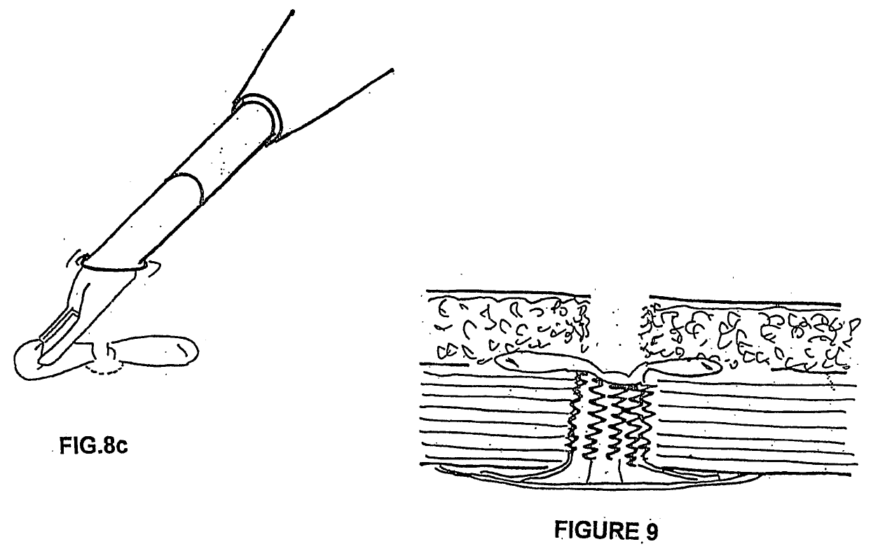

FIG. 5a depicts the improper positioning of the plate by excessive upward pulling of the ears. FIG. 7b shows its proper positioning.

FIG. 8a, 8b, 8c: The placement of the double earpiece (3) into the subcutaneous tissue, and its fixation.

FIG. 9: The final appearance of the plug and mesh in the trocar hole.

What is claimed is:

1. It is the plug and mesh that is to be used for the repair of large trocar wounds; it is formed from three pieces that;
a. is the intraabdominal mesh plate (1) that it is made of polypropylene, and that its obverse face is covered by either hydrophilic absorbable collagen barrier antiadhesive or ePTFE, that it is round in shape, and that it is laid between the abdominal wall and the intestines centering the inner trocar hole.
b. is the shaggy neck piece (2) that it is made of polypropylene, that it connects the intraabdominal mesh plate (1) to the double-ear piece (3), that it has a shaggy or thorny outer surface, that it fits into the trocar hole, that it is cylindrical in shape, and that it gets a better hold due to its shaggy shape.
c. is the double-ear piece (3) that it is made of polypropylene, that its two ears extend in opposite directions from the shaggy neck piece (2), that it has a shape of a propeller, and that it is fixated to the outer layer of the fascia.

2. It is the shaggy neck piece (2) that it is connected to claim 1, that its bottom opens up at a certain angle to form four legs, all of which unite with the intraabdominal mesh plate.

3. It is the double ear piece in connection with claim 1 that it has a concave shape at its radix, but becomes flat laterally.

4. It is the double-ear piece in connection with claim 1 that it has a parabolic angling at its vertical section, and that every ear of it has a certain angle with the cylindrical body.

5. It is the double-ear piece in connection with claim 1 that it has a parabolic angling at its vertical section, and that every ear of it has a certain angle with the cylindrical body.
